(19) United States
(12) Patent Application Publication
(10) Pub. No.: US 2021/0295729 A1
(43) Pub. Date: Sep. 23, 2021

(54) TRAINING SYSTEM FOR ENDOSCOPE MEDIUM

(71) Applicant: OLYMPUS CORPORATION, Tokyo (JP)

(72) Inventors: Tomoya SAKAI, Hachioji-shi (JP); Kosuke KISHI, Mitaka-shi (JP)

(73) Assignee: OLYMPUS CORPORATION, Tokyo (JP)

(21) Appl. No.: 17/202,614

(22) Filed: Mar. 16, 2021

Related U.S. Application Data
(63) Continuation of application No. PCT/JP2018/034397, filed on Sep. 18, 2018.

(51) Int. Cl. G09B 9/00; G16H 20/40; G16H 30/40; G16H 10/60; G09B 19/24

(52) U.S. Cl. CPC G09B 9/00; G16H 20/40; G09B 19/24; G16H 10/60; G16H 30/40

(57) ABSTRACT

This medical training system includes an operation unit, a display displaying an image data; a storage recording simulation data including biological data for forming a virtual manipulation space and device information about a first and second virtual medical devices, the simulation data using to generate image data for displaying the motion image on the display; and a controller configured to generate the image data based on the simulation data and an operation command input from the operation unit. The controller is configured to acquire the simulation data from the storage, calculate a first operation result of the first virtual medical device based on the simulation data and the operation command, calculate a second operation result of the second virtual medical device based on the first operation result, generate the image data based on the second operation result, and transmit the image data including the virtual manipulation space, and the first or the second virtual medical device to the display.

The present invention relates to a training system for an endoscope used in operation of medical devices. This application is a continuation application based on PCT Patent Application No. PCT/JP2018/034397, filed Sep. 18, 2018, the content of which is incorporated herein by reference.

### FIELD OF THE INVENTION

### Description of Related Art

Biopsy and manipulation (hereinafter referred to as simply “manipulation” including biopsy) using an endoscope and an endoscope system in which an endoscope treatment tool is inserted into a treatment tool insertion channel provided in an endoscope insertion part are minimally invasive and thus have been frequently performed in recent years. In a manipulation using such an endoscope, a plurality of operators, such as an operator who operates the endoscope treatment tool and an assistant who operates the endoscope, operate the endoscope and each operating part of the endoscope treatment tool while checking an image of a target part captured by an imaging unit of the endoscope to operate the endoscope and a treatment tool inside the body of a patient. In such a manipulation, all operators need to operate instruments in cooperation.

To perform a safe and efficient manipulation using an endoscope, training operators in a cooperative operation is important. Accordingly, training systems for training a plurality of operators in a technique of a cooperative operation have been proposed (Republication WO 2007/018289, and Japanese Unexamined Patent Application, First Publication No. 2013-6025).

### SUMMARY OF THE INVENTION

A medical training system according to a first aspect of the present invention includes: an operation unit; a display which is configured to display an image data; a storage which is configured to record simulation data including biological data for forming a virtual manipulation space and device information about a first virtual medical device set as a training target by the operation unit and a second virtual medical device that is a cooperative operation target with respect to the first virtual medical device; and a controller which is configured to generate the image data based on the simulation data and an operation command input from the operation unit; and, wherein the controller is configured to: acquire the simulation data from the storage; calculate a first operation result of the first virtual medical device based on the simulation data and the operation command; calculate a second operation result of the second virtual medical device based on the first operation result; generate the image data based on the second operation result; and transmit the image data including the virtual manipulation space, and the first virtual medical device or the second virtual medical device to the display.

In a second aspect of the present invention, in the medical training system according to the first aspect, the simulation data may include standard motion information about standard motions of the first virtual medical device and the second virtual medical device and cooperative operation information including degrees of influence of an operation of the first virtual medical device and an operation of the second virtual medical device, and the controller may be configured to calculate the second operation result based on the first operation result, the standard motion information, and the cooperative operation information.

In a third aspect of the present invention, in the medical training system according to the first aspect, the storage may be configured to record training result information that is results of execution of training of the first virtual medical device.

In a fourth aspect of the present invention, in the medical training system according to the first aspect, the first virtual medical device may include an endoscope and an endoscope treatment tool configured to use with an endoscope, and the controller may be configured to generate the image data including a virtual visual-field image of the endoscope and a virtual image of the endoscope treatment tool at a distal end of an endoscope insertion part of the endoscope.

In a fifth aspect of the present invention, in the medical training system according to the first aspect, the simulation data may include the standard biological data and preoperation examination data on a target patient on whom a manipulation will be performed, and the controller may be configured to generate the image data based on the standard biological data and the preoperation examination data.

In a sixth aspect of the present invention, in the medical training system according to the first aspect, the controller may be configured to calculate the second operation result based on the simulation data and the first operation result.

In a seventh aspect of the present invention, in the medical training system according to the first aspect, the controller may be configured to generate the image data based on the simulation data, the first operation result, and the second operation result.

In an eighth aspect of the present invention, in the medical training system according to the first aspect, the first virtual medical device may be the endoscope treatment tool, and the second virtual medical device is the endoscope.

In a ninth aspect of the present invention, in the medical training system according to the first aspect, the first virtual medical device may be the endoscope, and the second virtual medical device is the endoscope treatment tool.

In a tenth aspect of the present invention is a controller configured to calculate first operation result of a first virtual medical device based on a data regarding a biological tissue to form a virtual manipulation space, a device data regarding a first virtual medical device set as a training target by an operation unit and a second virtual medical device that is a cooperative operation target with respect to the first virtual medical device, and an operation command input from the operation unit; calculate second operation result of the second virtual medical device based on the first operation result; and generate an image data including the virtual manipulation space, and the first virtual medical device or the second medical device based on the second operation result.

In an eleventh aspect of the present invention, in the controller according to the tenth aspect, the controller may be configured to calculate the second operation result based on the first operation result, standard motion information about standard motions of the first virtual medical device and the second virtual medical device and cooperative operation information including degrees of influence of an operation of the first virtual medical device and an operation of the second virtual medical device.

In a twelfth aspect of the present invention, in the controller according to the tenth aspect, the first virtual medical device may include an endoscope treatment tool configured to use with an endoscope, and the controller may be configured to generate the image data including a virtual visual-field image of the endoscope and a virtual image of the endoscope treatment tool at a distal end of an endoscope insertion part of the endoscope.

In a thirteenth aspect of the present invention, in the controller according to the tenth aspect, the controller may be configured to generate the image data based on the standard biological data and the preoperation examination data.

In a fourteenth aspect of the present invention, in the controller according to the tenth aspect, the controller may be configured to calculate the second operation result based on the device data and the first operation result.

In a fifteenth aspect of the present invention, in the controller according to the tenth aspect, the controller may be configured to generate the image data based on the device data, the first operation result, and the second operation result.

In a sixteenth aspect of the present invention, in the controller according to the tenth aspect, the data regarding the biological tissue may include a data regarding body cavity and a data regarding lesion.

In a seventeenth aspect of the present invention is a medical training system including: an operation unit; a display which is configured to display an image data; and a controller configured to generate the image data. The controller is configured to make a first virtual medical device to operate in a virtual manipulation space based on an operation command input from the operation unit, the first virtual medical device being set to a training target for the operation unit; make a second virtual medical device to operate in the virtual manipulation space based on an operation of the first virtual medical device, the second virtual medical device being a cooperative operation target with respect to the first virtual medical device, and configured to generate the image data of the virtual manipulation space, and the first virtual medical device or the second virtual medical device.

In an eighteenth aspect of the present invention, in the medical training system according to the seventeenth aspect, the controller may be configured to make the second virtual medical device to operate in the virtual manipulation space based on an information of a biological tissue forming the virtual manipulation space and an information of a type of the second virtual medical device.

In a nineteenth aspect of the present invention, in the medical training system according to the seventeenth aspect, the controller may be configured to generate the image data based on an information of a biological tissue forming the virtual manipulation space and an information of types of the first virtual medical device and the second virtual medical device, and an information of operations of the first virtual medical device and the second virtual medical device.

In a twentieth aspect of the present invention, in the medical training system according to the seventeenth aspect, the controller may be configured to make the second virtual medical device in the virtual manipulation space based on a standard motion information about standard motions of the first virtual medical device and the second virtual medical device and cooperative operation information including degrees of influence of an operation of the first virtual medical device and an operation of the second virtual medical device.

### BRIEF DESCRIPTION OF THE DRAWINGS

FIG. 1 is a block diagram of a medical training system according to a first embodiment of the present invention.

FIG. 2 is a schematic diagram illustrating an example of a virtual medical device of the first embodiment of the present invention.

FIG. 3 is a block diagram of a training system for an endoscope according to the first embodiment of the present invention.

FIG. 4 is a flowchart illustrating processing of a controller in the medical training system according to the first embodiment of the present invention.

FIG. 5 is a flowchart illustrating processing of the controller at the time of initialization illustrated in FIG. 4.

FIG. 6 is a schematic diagram illustrating an operation state example of the medical training system according to the first embodiment of the present invention.

FIG. 7 is a schematic diagram illustrating a display image example of the medical training system according to the first embodiment of the present invention.

FIG. 8 is a schematic diagram illustrating a display image example of the medical training system according to the first embodiment of the present invention.

FIG. 9 is a schematic diagram illustrating a display image example of the medical training system according to the first embodiment of the present invention.

### DETAILED DESCRIPTION OF THE PREFERRED EMBODIMENTS

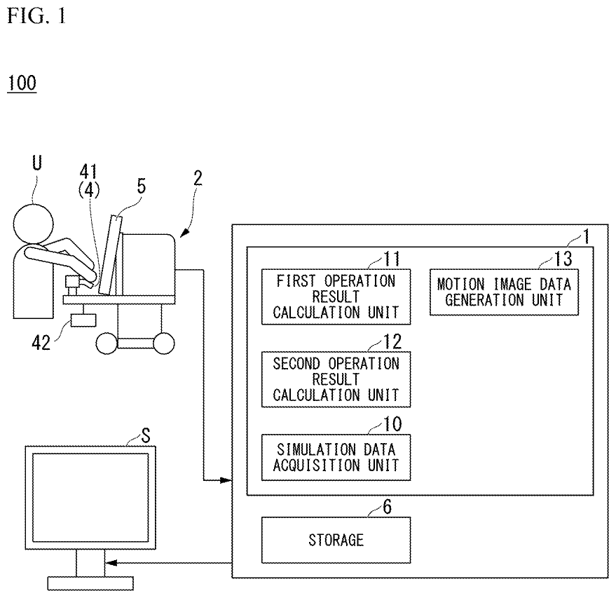

Hereinafter, an embodiment of a medical training system, a controller, and a recording medium according to the present invention will be described with reference to FIG. 1 to FIG. 9. FIG. 1 is a block diagram of a medical training system 100 (hereinafter referred to as a “training system”) according to the present embodiment.

The training system 100 is a system for training each operator to perform an operation in a manipulation in which a plurality of operators intensively operates a plurality of medical devices in cooperation. The training system 100 is a system for virtually operating a plurality of medical devices on a display. The training system 100 described in the present embodiment is an example in which a manipulation using an endoscope and an endoscope treatment tool as medical devices is performed while being displayed on a display 5. In addition, in the present embodiment, an example in which an operator who operates the endoscope treatment tool performs training is represented.

As illustrated in FIG. 1, the training system 100 includes an operation unit 4, the display 5, a storage 6, and a controller 1.

The operation unit 4 is an operation device for training in an operation of a medical device. The operation device imitates an operation unit of a real medical device. Unlike a real endoscope treatment tool and endoscope, the operation unit 4 does not include an operated unit. The operation unit 4 includes an operation input unit 41. The operation input unit 41 is configured such that operation inputs corresponding to an operation of bending a plurality of multiple-degree-of freedom joints provided in a bent part of a real treatment tool, an operation of advancing and retracting a treatment part, an operation of opening/closing the treatment part, and an operation corresponding to power feeding, and the like is capable of inputting thereto, for example.

Variety of components, for example, a trackball, a touch panel, a joystick, a master arm, or the like, or various known mechanisms in which a button, a lever, and the like are appropriately combined with these components may be employed as the operation input unit 41.

The operation input unit 41 includes a determinator 42 that determines an operation input amount. The determinator 42 may be, for example, an encoder or a sensor and determine movement angles, movement distances, movement speeds, and the like of various mechanisms constituting the operation input unit 41. An operation input signal is generated in response to a result of determination of an operation input amount of the operation input unit 41 obtained by the determinator 42. The generated operation signal is configured to be output to the controller 1.

The display 5 is provided in proximity to the operation unit 4. The display 5 displays a virtual treatment target part (hereinafter referred to as a “treatment target part”) and a virtual endoscope treatment tool as virtual captured images of an endoscope. Although this will be described later in detail, an operation input of the operation input unit 41 is cooperated with a movement of the treatment tool displayed on the display 5. A user U can operate the operation input unit 41 while checking a virtual image of a medical device displayed on the display 5. Although FIG. 1 illustrates an example in which a plurality of displays 5 are provided, the training system may include at least one display 5 that is capable of being checked by the user U.

The storage 6 records simulation data. The simulation data is data used to generate image data to be displayed on the display 5 and is data for generating a motion image in a virtual manipulation.

The storage 6 may be configured as a hard disk drive, a solid state drive, a volatile memory, a cloud via a wired or wireless network, or a combination thereof.

The simulation data includes various types of data about a manipulation that requires a cooperative operation as a training target. For example, the simulation data may include task data, standard biological data, treatment tool data (device information), a visual-field image of an endoscope, and standard motion information.

The task data includes a manipulation type, an attainment target in each manipulation, and procedure data about a procedure necessary to reach the attainment target. As examples of manipulation type, an endoscopic submucosal dissection (ESD) task, an endoscopic mucosal resection (EMR) task, a suture task, and an incision task are conceivable. As an attainment target, for example, resecting a tumor in a lumen in the ESD task and suturing an opened part in a lumen in the suture task are conceivable. As procedure data, for example, a three-dimensional positions of the piercing point and piercing out point of a suture needle, and the size of the suture needle are conceivable in the suture task.

The standard biological data is data about a biological tissue that is a training target. As examples of the standard biological data, lumen-shaped polygon data, physical property data such as the elasticity and thickness of a lumen, physical property data such as the size of a tumor (width, depth, and thickness), a three-dimensional position of the center of the tumor, and physical property data such as elasticity of the tumor, and the like are conceivable.

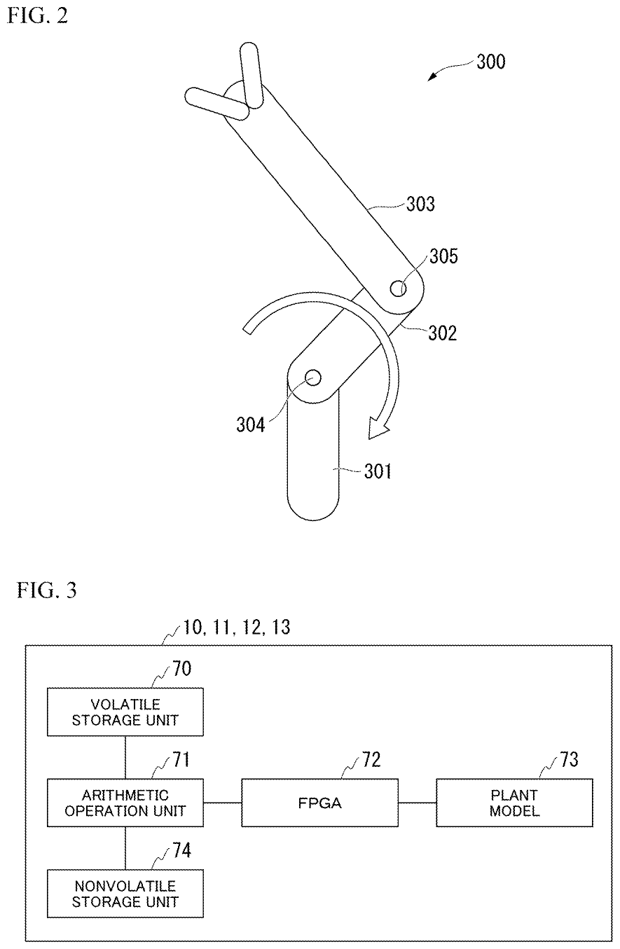

The treatment tool data is various types of data about a virtual medical device that is a training target. The treatment tool data includes device information about a first virtual medical device set as a training target by the operation unit 4 and a second virtual medical device that is a cooperative operation target with respect to the first virtual medical device. Examples of the treatment tool data include a type and a model number of a treatment tool, and the like. A specific example of treatment tool data is represented. For example, when a virtual medical device is a polyarticular treatment tool 300 illustrated in FIG. 2, treatment tool data is the shape and dimensions of the treatment tool 300, positions of joints 304 and 305, and a subordinate relationship of links 301 and 303. More specifically, data such as the fact that the links 301 and 303 have columnar shapes, the fact that the first joint 304 is positioned at the distal end of the first link 301 and the second joint is positioned at the distal end of the second link 302, and the fact that the second link 302 is subordinate to the first joint 304 is conceivable.

The treatment tool data includes standard motion information. The standard motion information is data about a motion of each part of a treatment tool that is a virtual device which corresponds to a motion amount of the operation unit on the basis of relation data of the motion amount of the operation unit set in a real medical device and a movement amount of the treatment tool. For example, when the user U has rotated a first joint operation unit of the operation unit 4 by an angle of 1 degree clockwise, the standard motion information may be information about a rotating joint among a plurality of joints of the treatment tool 300, the rotating direction and the rotating amount of the treatment tool 300. The standard motion information may be, for example, information representing that the first joint operation unit of the operation unit 4 rotates clockwise and information representing that a movement amount of the treatment tool 300 is twice an operation amount of the operation unit 4.

The simulation data includes cooperative operation information. The cooperative operation information includes data about degrees of influence of an operation of the first virtual medical device and an operation of the second virtual medical device. This will be described in detail later.

The storage further records training result information. The training result information is results of training performed in the first virtual medical device that is a training target.

The controller 1 includes a simulation data acquisition unit 10, a first operation result calculation unit 11, a second operation result calculation unit 12, and a motion image data generation unit 13.

As illustrated in FIG. 3, the simulation data acquisition unit 10, the first operation result calculation unit 11, the second operation result calculation unit 12, and the motion image data generation unit 13 include an arithmetic operation unit 71 such as a CPU, a volatile storage unit 70, a nonvolatile storage unit 74, a field-programmable gate array (FPGA) 72, and a plant model 73.

For example, a RAM or the like can be used as the volatile storage unit 70. For example, a flash memory or the like can be used as the nonvolatile storage unit 74. The FPGA 72 is a gate array in which details of a program can be updated. The arithmetic operation unit 71 is connected to the volatile storage unit 70, the nonvolatile storage unit 74, and the FPGA 72.

The plant model 73 is data, in which a structure, a dimension, and operating states of virtual operated units, a virtual driving unit, and a virtual driving unit driver and the like of an endoscope and an endoscope treatment tool, are physically modeled, and is stored in a recording medium and like.

Motion signal generation data for generating a motion signal of the virtual driving unit driver is stored in the FPGA 72 on the basis of an operation signal output from the operation input unit 41. The motion signal generation data includes a signal generation program for generating the motion signal, a control parameter, and the like. When an input operation signal is received from the operation input unit 41, the arithmetic operation unit 71 executes simulations of operations of the virtual driving unit driver and the virtual driving unit and operations of the virtual operated units with reference to data of the plant model 73.

Each component included in the controller 1 may be configured such that each or all of the components are realized by a computer including one or more processors, a logic circuit, a memory, an input/output interface, a computer-readable recording medium, and the like. In this case, programs for realizing functions of each component or the entire controller are recorded in the recording medium. The simulation may execute by making a computer to read and to realize the recorded program. For example, the processor may be at least one of a CPU, a digital signal processor, and a graphics processing unit (GPU). For example, the logic circuit may be at least one of an application specific integrated circuit (ASIC) and a field-programmable gate array (FPGA).

For example, the aforementioned various functions and processing in the training system 100 may be performed by a computer system reading and executing a program for realizing functions and processing of the simulation data acquisition unit 10, the first operation result calculation unit 11, the second operation result calculation unit 12, the motion image generation unit 13, and the like illustrated in FIG. 1, which is recorded in a computer-readable recording medium. The “computer system” referred to here may include an OS and hardware such as peripheral devices. In addition, the “computer system” also includes a homepage providing environment (or a display environment) if a WWW system is used. Further, “computer-readable recording medium” refers to a writable nonvolatile memory such as a flexible disk, a magneto-optic disc, a ROM, or a flash memory, a portable medium such as a CD-ROM, and a storage device such as a hard disk embedded in a computer system.

The “computer-readable recording medium” may also be a recording medium storing a program for a specific time, such as a volatile memory (e.g., a dynamic random access memory (DRAM)) in a computer system serving as a server or a client when the program is transmitted through a network such as the Internet or a communication circuit such as a telephone circuit. The aforementioned program may be transmitted from a computer system in which this program is stored in a storage device to other computer systems through a transmission medium or transmitted waves in the transmission medium. Here, the “transmission medium” that transmits the program is a medium having a function of transmitting information, for example, a network (communication network) such as the Internet, and a communication circuit (communication line) such as a telephone circuit. The aforementioned program may be a program for realizing some of the above-described functions. The aforementioned program may be a program that realizes the above-described functions by being combined with a program that has already been recorded on a computer system, known as a difference file (different program).

The simulation data acquisition unit 10 acquires standard biological data that forms an image data of a virtual manipulation space and device information about the first virtual medical device and the second virtual medical device that is a cooperative operation target from the storage 6.

The first operation result calculation unit 11 calculates a first operation result that is a result obtained by performing a motion simulation of the first virtual medical device on the basis of the acquired simulation data (the standard biological data and the device information) and an operation command from the operation input unit 41. Specifically, a movement amount of the operation input unit 41, such as an operation amount and an operation direction of the operation input unit 41 are determined by the determinator 42 and a movement state of the medical device is calculated on the basis of a determination result and the device information acquired from the storage 6.

The second operation result calculation unit 12 calculates a second operation result on the basis of the first operation result and the device information acquired from the storage 6. The second operation result is obtained by performing a motion simulation of the second virtual medical device and corresponding to the first operation result. For example, the endoscope and the endoscope treatment tool may each affect operations the other. Accordingly, an operation result of the second virtual medical device which corresponds to the first operation result is calculated in order to generate image data.

The motion image data generation unit 13 performs a simulation of a state in which the first virtual medical device and the second virtual medical device operate in a virtual manipulation space on the basis of the device information acquired from the storage 6, the standard biological data, the first operation result, and the second operation result, and generates image data for displaying a simulation result on the display 5.

The controller 1 includes an operation command reception unit that receives an operation command signal from the operation unit 4, and a transmission unit that transmits various signals to the operation unit 4 and the display 5.

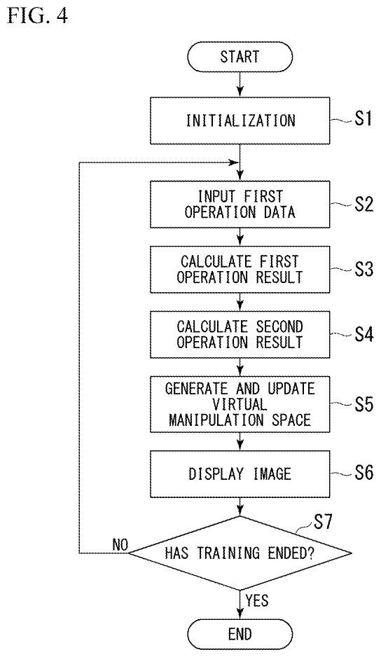

Next, the operation of the training system 100 will be described. FIG. 4 is a flowchart illustrating processing of the controller 1 in the medical training system 100.

First, initialization is performed (step S1). FIG. 5 is a flowchart illustrating processing of the controller 1 at the time of initialization S1.

At the time of initialization S1, a menu screen is displayed on the display 5 and the user U selects a manipulation that is a training target, a medical device, a treatment target part, and the like using the operation input unit 41. First, the user U sets a task of a training target, for example, an ESD task or a suture task, by operating the operation input unit 41 while viewing the menu screen displayed on the display 5 (step S11). For example, the ESD task may be set.

Next, the user U sets standard biological data that is a treatment target part by operating the operation input unit 41 while viewing the menu screen displayed on the display 5 (step S12). For example, a colon may be set as a target part on which the ESD task will be performed.

Next, the user U sets treatment tool data by operating the operation input unit 41 while viewing the menu screen displayed on the display 5 (step S13). For example, a type of a treatment tool (gripping forceps or the like) to be used for colon ESD may be selected from a treatment tool list and set.

Next, the user U sets a virtual manipulation space by operating the operation input unit 41 while viewing the menu screen displayed on the display 5 (step S14). For example, the position and the size of a tumor in the colon and the like may be set.

Operation commands based on the operation inputs of the operation input unit 41 performed in steps S11 to S14 are transmitted to the controller 1.

The controller 1 acquires simulation data such as the manipulation of the training target and the medical device from the storage on the basis of the received operation commands. The controller 1 generates image data on the basis of the acquired simulation data.

Specifically, the controller 1 acquires device information, standard biological data about a colon lumen and a tumor, and an evaluation index. The device information is, for example, information about an endoscope, a treatment tool (e.g., a gripping forceps), and an over-tube. The evaluation index indicates, for example, a distance between the colon lumen and the medical device. The controller 1 generates image data of the virtual manipulation space on the basis of the acquired data. The medical device and the colon lumen are defined as polygon data and the position of the tumor and the evaluation index are defined as physical numerical values.

The controller 1 transmits the generated image data of the virtual manipulation space to the display 5 and displays an initial image of the virtual manipulation space on the display 5. Initialization S1 of training ends.

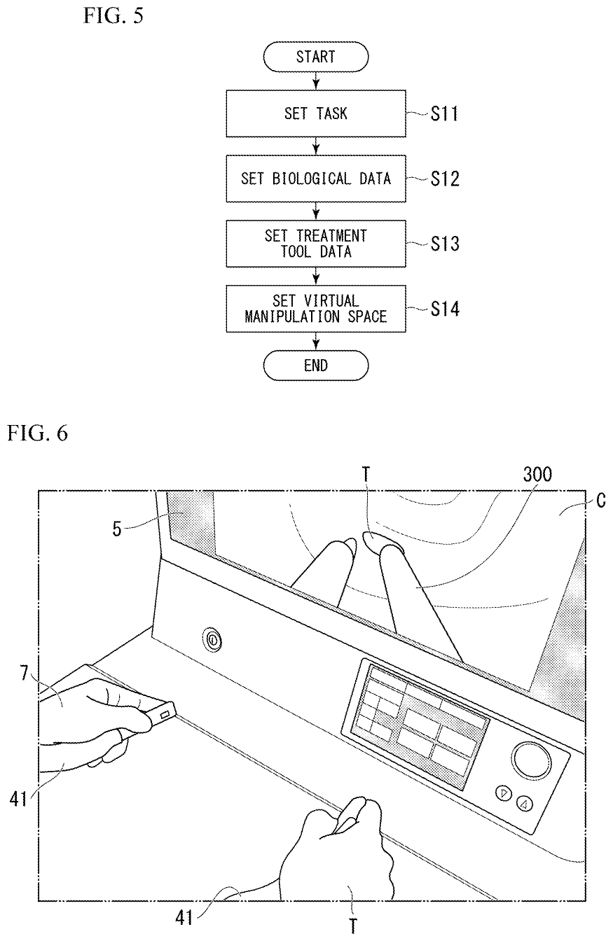

Subsequently, the user U starts training. The user U inputs first operation data by operating the operation input unit 41 (step S2). For example, a virtual image of the tumor T and the treatment tool 300 in the colon C that is the virtual manipulation space is displayed as an initial image, as illustrated in FIG. 6. The user U operates the operation input unit 41 while checking the initial image displayed on the display 5. The operation amount, the operation direction, and the like of the operation input unit 41 are determined by the determinator 42 and transmitted to the controller 1 as first operation data.

Upon reception of the first operation data, the controller 1 calculates a first operation result, such as a movement amount of the treatment tool 300, corresponding to the first operation data through simulation processing (step S3).

Next, the controller 1 calculates a second operation result. An ideal operation state of the second virtual medical device is calculated on the basis of the first operation result, the device information, and an operation index included in the simulation data. The operation index is an index set as a specific condition desired for the operation of the second virtual medical device on the basis of an operation state desired in a real manipulation. An exemplary example of the operation index will be shown below.

### Operation Index Example 1

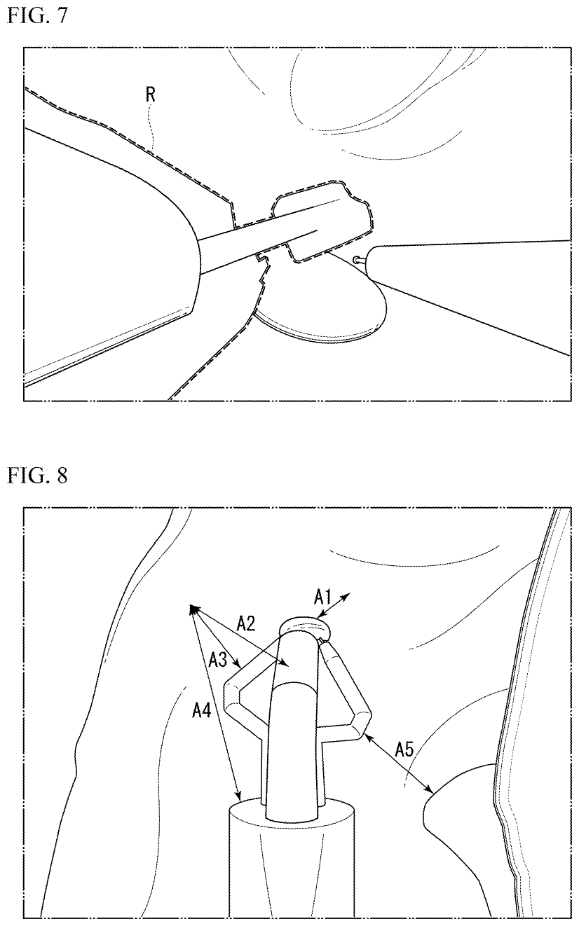

A ratio B₁ of a region hidden by the treatment tool in an endoscope visual-field image (an area in which the treatment tool is imaged in the endoscope visual field, a region R indicated by a dotted line in FIG. 7) to the whole area of the endoscope visual-field image is set to be equal to or less than 30%. It is desirable that the ratio B₁ be lower because the lumen is sufficiently viewed in the endoscope visual-field image.

### Operation Index Example 2

A distance B₂ between the treatment target part and the distal end of the treatment tool at the time of treatment, for example, a distance of an arrow A1 illustrated in FIG. 8, is set to be equal to or less than 20 mm. It is desirable that the distance B₂ be shorter because the treatment tool can be positioned more accurately.

### Operation Index Example 3

A shortest distance B₃ between the treatment tool and surrounding tissues (intestinal wall), for example, distances of arrows A2 to A5 illustrated in FIG. 8, is set to be equal to or greater than 5 mm. It is desirable that the distance B₃ be longer because it is difficult for the treatment tool to collide with the intestinal canal.

The exemplified operation index examples 1 to 3 may be additionally weighted for each evaluation index, as represented by the following mathematical expression (1), and the sum of weighted evaluation indexes may be set to an operation function and used to calculate the second operation result. Weighting can be arbitrarily set by the user U. Accordingly, each user U is capable of appropriately setting conditions to be considered important at the time of training.

$$\begin{matrix}[ Math . 1 ] & \\ \begin{matrix}S ( p , q ) = \sum {w}_{i} {B}_{i} \\ = {w}_{1} {B}_{1} + {w}_{2} {B}_{2} + …\end{matrix} & ( 1 )\end{matrix}$$

p: first operation result

q: second operation result

S(p, q): operation function

Wi: weight for each operation index

Bi: evaluation point of operation index

Next, the controller 1 generates a virtual manipulation space on the basis of the calculated second operation result (step S5). Image data of an initial screen that has already been generated is updated on the basis of the first operation result and the second operation result in the virtual manipulation space. The updated image data is transmitted from the controller 1 to the display 5 and the image displayed on the display 5 is updated (step S6). After step S6, the controller 1 determines whether training ends (step S7), and step S2 to step S6 are repeated at any time in response to the operation of the operation input unit 41 until training end is determined.

When the virtual manipulation space is updated (step S5), the second operation result may be calculated using cooperative operation information in addition to the aforementioned one. The cooperative operation information is an index including degrees of influence of the operation of the first virtual medical device and the operation of the second virtual medical device.

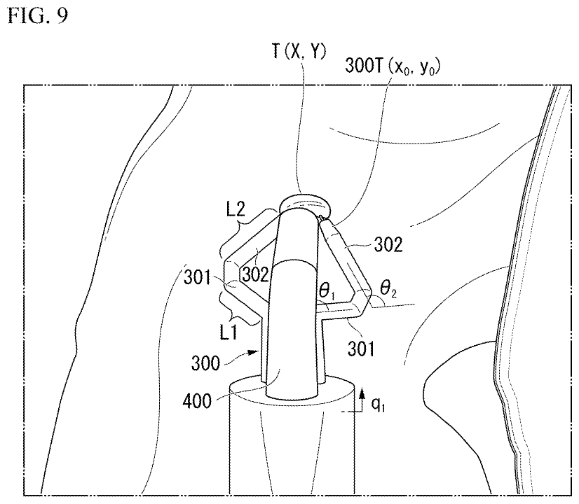

A case in which the second operation result is calculated using only the distance between the treatment target part and the distal end of the treatment tool at the time of treatment using the treatment tool will be exemplified with reference to FIG. 9. As illustrated in FIG. 9, coordinates indicating the position of the tumor T are set to T(X, Y). Coordinates 300T(X₀, Y₀) of the distal end 300T of the treatment tool 300 is calculated using the following mathematical expressions (2) and (3) on the basis of the length L1 of a first arm 301 of the treatment tool 300, the length L2 of a second arm 302, an angle θ1 of the first arm 301 with respect to an endoscope 400, and an angle θ2 of the second arm 302 with respect to the first arm 301.

[Math. 2]

X₀=L₁ sin(θ₁)+L₂ sin(θ₁−θ₂) (2)

[Math. 3]

Y₀=L₁ cos(θ₁)+L₂ cos(θ₁−θ₂)+q₁ (3)

In mathematical expression (3), q₁ is a previous second operation result. A rate of change ΔS in the distance between the tumor T and the distal end 300T of the treatment tool 300 is calculated using the position T(X, Y) of the tumor T and the position 300T(X₀, Y₀) of the distal end 300T of the treatment tool 300. A ratio ΔS/Δq₁ of the calculated rate of change ΔS to an amount of change Δq₁ in the second operation result is represented as a function using a previous first operation result p1 and a present first operation result p2. When values θ₁₀, θ₂₀ and q₁₀ of the previous second operation result are inserted into the ratio ΔS/Δq₁, a new second operation result is obtained. The rate of change in the posture of the treatment tool 300 before and after change in the first operation result can be calculated using this new second operation result. The calculated rate of change in the posture of the treatment tool 300 may be used as the second operation result.
According to the training system 100 in the present embodiment, a virtual movement state of the second virtual medical device that is a cooperative operation target is reproduced and displayed on the display 5 on the basis of an operation result of the first virtual medical device. Since the second operation result is calculated on the basis of the first operation result and device information acquired from simulation data, a more realistic virtual manipulation space is formed. As a result, each operator is capable of individually performing a manipulation training that requires a cooperative operation of a plurality of operators. Accordingly, a plurality of operators is capable of performing training with high efficiency without needing to simultaneously perform training.

Although an example in which the user U uses the endoscope treatment tool as the first virtual medical device has been illustrated in the above-described embodiment, a virtual manipulation space is capable of being formed in the same manner even if the endoscope is used as the first virtual medical device as a training target and the endoscope treatment tool is used as the second virtual medical device.

Although one embodiment of the present invention has been described above, the technical scope of the present invention is not limited to the aforementioned embodiment and each component may be modified in various manners or deleted, or components of the embodiment may be combined without departing from essential characteristics of the present invention.

### First Modified Example

A first modified example of the training system 100 according to the aforementioned embodiment will be described. In the following description, the same parts as components in the aforementioned embodiment are denoted by the same signs and description thereof is omitted. The training system 100 of the present modified example is an example in which training result information is additionally recorded in the storage 6. The training result information is a result of execution of training of the first virtual medical device and is recorded in the storage when training ends. A configuration in which a virtual manipulation space is formed on the basis of the recorded training result information may be employed.

For example, results of separately performed endoscope training may be recorded as training result information in the storage. Data about an operation tendency (habit) during a user operation is accumulated according to the recorded training result information. As a result, a specific simulation is capable of being experienced in advance with respect to a cooperative operation of an actual manipulation if training result information obtained by an operator different from a training target person has been recorded when the actual manipulation is performed, for example.

### Second Modified Example

A training system of a second modified example will be described. Simulation data may further include preoperation information (preoperation examination data) on a target patient on which a manipulation will be performed. Preoperation information on a patient may be, for example, information on a preoperation examination performed on the patient before an operation. For example, when there is a CT image of a CT examination, preoperation information is added to standard biological data. Image data of a virtual manipulation space may be generated using the preoperation information and the standard biological data.

According to the present modified example, preoperation examination data of a specific patient is recorded in the storage in addition to standard biological data and thus a preoperation simulation of the specific patient may be performed. Here, even in the case of an operation that requires a cooperative operation, a simulation of the operation is capable of being performed with high efficiency because each operator can individually perform training.

### Third Modified Example

Although an example in which a virtual manipulation space closer to an operation state of a real medical device is displayed on the display 5 has been illustrated in the aforementioned embodiment, evaluation information for feedback of training results to the user U may be additionally displayed in addition to an image of the virtual manipulation space at the time of training. That is, a configuration in which optimal data that is simulation data about an optimal cooperative operation is recorded in the storage and the controller 1 compares the first operation result with the optimal data and displays comparison results on the display 5 may be employed.

Although an example in which the endoscope is used has been illustrated in the aforementioned embodiment, a medical device as a training target is not limited thereto. For example, a laparoscope, a manipulation robot, and the like may be used. Regarding manipulations, the present invention may be implemented in manipulations performed in an internal medicine department and a surgery department.

What is claimed is:

1. A medical training system comprising:
an operation unit;
a display which is configured to display an image data;
a storage which is configured to record simulation data including biological data for forming a virtual manipulation space and device information about a first virtual medical device set as a training target by the operation unit and a second virtual medical device that is a cooperative operation target with respect to the first virtual medical device; and
a controller which is configured to generate the image data based on the simulation data and an operation command input from the operation unit; and,
wherein the controller is configured to:
acquire the simulation data from the storage;
calculate a first operation result of the first virtual medical device based on the simulation data and the operation command;
calculate a second operation result of the second virtual medical device based on the first operation result;
generate the image data based on the second operation result; and
transmit the image data including the virtual manipulation space, and the first virtual medical device or the second virtual medical device to the display.

2. The medical training system according to claim 1, wherein the simulation data includes standard motion information about standard motions of the first virtual medical device and the second virtual medical device and cooperative operation information including degrees of influence of an operation of the first virtual medical device and an operation of the second virtual medical device, and
the controller is configured to calculate the second operation result based on the first operation result, the standard motion information, and the cooperative operation information.

3. The medical training system according to claim 1, wherein the storage is configured to record training result information that is results of execution of training of the first virtual medical device.

4. The medical training system according to claim 1, wherein the first virtual medical device includes an endoscope treatment tool configured to use with an endoscope, and
the controller is configured to generate the image data including a virtual visual-field image of the endoscope and a virtual image of the endoscope treatment tool at a distal end of an endoscope insertion part of the endoscope.

5. The medical training system according to claim 1, wherein the simulation data includes the standard biological data and preoperation examination data on a target patient on whom a manipulation will be performed, and
the controller is configured to generate the image data based on the standard biological data and the preoperation examination data.

6. The medical training system according to claim 1, wherein the controller is configured to calculate the second operation result based on the simulation data and the first operation result.

7. The medical training system according to claim 1, wherein the controller is configured to generate the image data based on the simulation data, the first operation result, and the second operation result.

8. The medical training system according to claim 1, wherein the first virtual medical device is the endoscope treatment tool, and the second virtual medical device is the endoscope.

9. The medical training system according to claim 1, wherein the first virtual medical device is the endoscope, and the second virtual medical device is the endoscope treatment tool.

10. A controller configured to calculate first operation result of a first virtual medical device based on a data regarding a biological tissue to form a virtual manipulation space, a device data regarding a first virtual medical device set as a training target by an operation unit and a second virtual medical device that is a cooperative operation target with respect to the first virtual medical device, and an operation command input from the operation unit;
calculate second operation result of the second virtual medical device based on the first operation result; and
generate an image data including the virtual manipulation space, and the first virtual medical device or the second virtual medical device based on the second operation result.

11. The controller according to claim 10, wherein
the controller configured to calculate the second operation result based on the first operation result, standard motion information about standard motions of the first virtual medical device and the second virtual medical device and cooperative operation information including degrees of influence of an operation of the first virtual medical device and an operation of the second virtual medical device.

12. The controller according to claim 10, wherein
the first virtual medical device includes an endoscope treatment tool configured to use with an endoscope, and
the controller is configured to generate the image data including a virtual visual-field image of the endoscope and a virtual image of the endoscope treatment tool at a distal end of an endoscope insertion part of the endoscope.

13. The controller according to claim 10, wherein
the controller is configured to generate the image data based on the standard biological data and the preoperation examination data.

14. The controller according to claim 10, wherein
the controller is configured to calculate the second operation result based on the device data and the first operation result.

15. The controller according to claim 10, wherein
the controller is configured to generate the image data based on the device data, the first operation result, and the second operation result.

16. The controller according to claim 10, wherein
the data regarding the biological tissue includes a data regarding body cavity and a data regarding lesion.

17. A medical training system comprising:
an operation unit;
a display which is configured to display an image data; and
a controller configured to generate the image data;
wherein the controller is configured to:
make a first virtual medical device to operate in a virtual manipulation space based on an operation command input from the operation unit, the first virtual medical device being set to a training target for the operation unit;
make a second virtual medical device to operate in the virtual manipulation space based on an operation of the first virtual medical device, the second virtual medical device being a cooperative operation target with respect to the first virtual medical device; and
generate the image data of the virtual manipulation space and the first virtual medical device or the second virtual medical device.

18. The medical training system according to claim 17, wherein the controller is configured to make the second virtual medical device to operate in the virtual manipulation space based on an information of a biological tissue forming the virtual manipulation space and an information of a type of the second virtual medical device.

19. The medical training system according to claim 17, wherein the controller is configured to generate the image data based on an information of a biological tissue forming the virtual manipulation space and an information of types of the first virtual medical device and the second virtual medical device, and an information of operations of the first virtual medical device and the second virtual medical device.

20. The medical training system according to claim 17, wherein the controller is configured to make the second virtual medical device in the virtual manipulation space based on a standard motion information about standard motions of the first virtual medical device and the second virtual medical device and cooperative operation information including degrees of influence of an operation of the first virtual medical device and an operation of the second virtual medical device.
